(19) United States
(12) Patent Application Publication
(10) Pub. No.: US 2011/0251113 A1
(43) Pub. Date: Oct. 13, 2011

(54) LUBRICATING GREASE COMPOSITIONS

(76) Inventor: Stefan Daegling, Hamburg (DE)

(21) Appl. No.: 13/130,594

(22) PCT Filed: Nov. 24, 2009
(86) PCT No.: PCT/EP2009/065691

§ 371 Date: Jun. 24, 2011

(30) Foreign Application Priority Data
Nov. 24, 2008 (EP) 08169822.7

(51) Int. Cl. C10M 117/06; G01N 33/30

(52) U.S. Cl. 508/506; 73/10

(57) ABSTRACT

A Lubricating grease composition comprising a base oil and a thickener system comprising (a) a lithium soap of a C₁₂ to C₂₄ hydroxycarboxylic acid and (b) an alkaline earth metal salt of a C₂ to C₁₂ dicarboxylic acid. The lubricating grease compositions exhibit increased water resistance and shear stability together with excellent oil bleeding properties. The lubricating grease compositions also contain lower levels of lithium than are present in a conventional lithium based grease compositions.

### FIELD OF THE INVENTION

The present invention relates to lubricating grease compositions, particularly to lubricating grease compositions having improved water resistance, improved shear stability and excellent oil bleeding properties.

### BACKGROUND OF THE INVENTION

The primary purpose of lubrication is separation of solid surfaces moving relative to one another, to minimise friction and wear. The materials most frequently used for this purpose are oils and greases. The choice of lubricant is mostly determined by the particular application.

Lubricating greases are employed where heavy pressures exist, where oil drip from the bearings is undesirable or where the motion of the contacting surfaces is discontinuous so that it is difficult to maintain a separating film in the bearing. Because of design simplicity, decreased sealing requirements and less need for maintenance, greases are almost universally given first consideration for lubricating ball and roller bearings in electric motors, household appliances, automotive wheel bearings, machine tools, aircraft accessories and mining tools. Greases are also used for the lubrication of small gear drives and for many slow-speed sliding applications.

Lubricating greases are the lubricants of choice in a dual mass flywheel application. A dual mass flywheel eliminates excessive transmission gear rattle, reduces gear change/shift effort, and increases fuel economy. Dual mass flywheels are typically fitted to light-duty diesel trucks with standard manual transmissions and to higher performance luxury vehicles to dampen vibration in the drive train. This allows vehicles to be operated for longer periods without long term damage.

Lubricating greases consist primarily of a fluid lubricant, such as an oil, and a thickener. Essentially, the same type of oil is employed in compounding a grease as would normally be selected for oil lubrication. Fatty acid soaps of lithium, calcium, sodium, aluminium and barium are most commonly used as thickeners. Lithium complex soaps, where a lithium salt is reacted with an organic complexing agent e.g. azelaic acid, sebacic acid are also commonly used as thickeners in grease compositions.

Due to ever increasing demands for higher performance, it would be desirable to provide greases which exhibit improved lubrication properties, and in particular, high dropping points, improved water resistance, improved shear stability, reduced oil bleeding properties, as well as improved noise and density profile.

Further, since lithium metal is getting shorter in supply, it would be advantageous to provide a grease formulation which uses lower levels of lithium than is used in conventional lithium complex grease compositions.

### SUMMARY OF THE INVENTION

According to the present invention there is provided a lubricating grease composition comprising:
(i) base oil; and
(ii) a thickener system comprising (a) a lithium soap of a C₁₂ to C₂₄ hydroxycarboxylic acid and (b) an alkaline earth metal salt of a C₂ to C₁₂ dicarboxylic acid.

According to the present invention there is further provided the use of a lubricating composition as described hereinbelow for increasing water resistance.

According to the present invention there is further provided the use of a lubricating composition as described hereinbelow for increasing shear stability.

According to the present invention there is further provided the use of a lubricating composition as described hereinbelow for reducing oil bleeding.

According to the present invention there is further provided the use of a lubricating grease composition as described hereinbelow in a dual mass flywheel application.

It has surprisingly been found that the grease compositions of the present invention exhibit excellent water resistance, shear stability and oil bleeding properties, as well as excellent friction reducing properties, good stability, good wear properties, high resistance to centrifugal forces and increased grease lifetime, particularly in a mass flywheel application or coupling application. The grease compositions of the present invention are also advantageous from the viewpoint of containing lower levels of lithium than are used in a conventional lithium-based grease composition.

### DETAILED DESCRIPTION OF THE INVENTION

The lubricating grease of the present invention comprises, as an essential component, a base oil.

There are no particular limitations regarding the base oil composition used in the method according to the present invention, and various conventional mineral oils and synthetic oils may be conveniently used. For the purpose of this description, the term “base oil” is meant to also include a grease base stock.

Preferably, the lubricating composition comprises at least 30 wt. % base oil, preferably at least 50 wt. %, more preferably at least 70 wt. %, based on the total weight of the lubricating composition.

The base oil composition used in the present invention may conveniently comprise mixtures of one or more mineral oils and/or one or more synthetic oils.

The base oil for use herein preferably has a kinematic viscosity at 40° C. (according to ASTM D445) of from 10 to 2000 mm²/s.

Mineral oils include liquid petroleum oils and solvent-treated or acid-treated mineral lubricating oil of the paraffinic, naphthenic, or mixed paraffinic/naphthenic type which may be further refined by hydrofinishing processes and/or dewaxing.

Suitable base oils for use in the lubricating oil composition of the present invention are Group I, Group II or Group III base oils, polyalphaolefins, Fischer-Tropsch derived base oils and mixtures thereof.

By “Group I” base oil, “Group II” base oil and “Group III” base oil in the present invention are meant lubricating oil base oils according to the definitions of American Petroleum Institute (API) categories I, II and III. Such API categories are defined in API Publication 1509, 15th Edition, Appendix E, April 2002.

Suitable Fischer-Tropsch derived base oils that may be conveniently used as the base oil in the lubricating oil composition of the present invention are those as for example disclosed in EP 0 776 959, EP 0 668 342, WO 97/21788, WO 00/15736, WO 00/14188, WO 00/14187, WO 00/14183, WO 00/14179, WO 00/08115, WO 99/41332, EP 1 029 029, WO 01/18156 and WO 01/57166.

Synthetic oils include hydrocarbon oils such as olefin oligomers (PAOs), dibasic acid esters, polyol esters, and dewaxed waxy raffinate. Synthetic hydrocarbon base oils sold by the Shell Group under the designation “XHVI” (trade mark) may be conveniently used.

In a preferred embodiment of the invention, the base oil is that of mineral origin sold by the Royal Dutch/Shell Group of Companies under the designations “HVI” or “MVIN”.

In another embodiment of the invention the lubricating composition comprises a polyalkylene glycol base oil. There are no particular limitations regarding the polyalkylene glycol base oil used in lubricating compositions according to the present invention, and various conventional polyalkylene glycols may be conveniently used.

The polyalkylene glycols (PAG) used according to the invention may exhibit alkylene oxide units with 1 to 6 carbon atoms (—R—O—) as monomer units.

The polyalkylene glycols may exhibit hydrogen end groups, alkyl, aryl, alkylaryl, aryloxy, alkoxy, alkylaryloxy and/or hydroxy end groups. Alkylaryloxy groups should also be understood to mean arylalkyl (ene)oxy groups and alkylaryl groups to mean arylalkyl(ene) groups (e.g. aryl CH₂CH₂—). The end groups of the alkyl type, including the alkoxy type, or of the aryl types, including the alkylaryl type, aryloxy type and alkylaryloxy type preferably exhibit 6 to 24 carbon atoms, particularly preferably 6 to 18 carbon atoms, based on the aryl types, and preferably 1 to 12 carbon atoms, based on the alkyl types.

The polyalkylene glycols may be either homopolymers, namely polypropylene glycol (and/or polypropylene oxide) or copolymers, terpolymers etc. For the latter cases, the monomer units may exhibit a random distribution or a block structure. If the polyalkylene glycols are not homopolymers, preferably at least 20%, preferably at least 40% of all monomer units are producible from polypropylene oxide (PO), and also preferably, at least 20% of all monomer units of these polyalkylene glycols are producible by using ethylene oxide (EO) (PO/EO copolymers). According to a further embodiment, preferably at least 20%, preferably at least 40% of all monomer units are obtainable from butylene oxide (BO) and, moreover, preferably at least 20% of all monomer units of these polyalkylene glycols are obtainable by using ethylene oxide (BO/EO copolymers).

In preferred embodiments herein, preferably at least 50%, more preferably at least 80% of all monomer units are producible from propylene oxide, with the remainder producible from ethylene oxide.

In a particularly preferred embodiment herein, the polyalkylene glycols are homopolymers of propylene oxide. Suitable examples of polypropylene homopolymers are commercially available from Dow Chemicals under the tradename Synalox®, for example, Synalox® 100-150B.

When (poly)alcohols are used, the starting compound is incorporated into the polymer and, according to the meaning of the invention, also referred to as end group of the polymer chain. Suitable starting groups consist of compounds comprising active hydrogen such as e.g. water, n-butanol, propylene glycol, ethylene glycol, neopentyl glycols such as pentaerythritol, ethylene diamine, phenol, cresol or other (C₁ to C₁₆ (mono, di or tri)alkyl) aromatics, (hydroxyalkyl) aromatics, hydroquinone, aminoethanolamines, triethylenetetramines, polyamines, sorbitol or other sugars. Other C—H acidic compounds such as carboxylic acids or carboxylic anhydrides, can also be used as starting compounds. Other suitable starting compounds include longer chain alcohols, such as C₁₀ to C₁₈ alcohols.

Preferably, the polyalkylene glycols comprise aryl groups or corresponding heteroaromatic groups, e.g. inserted into the polymer chain, as side groups or end groups; the groups may, if necessary, be substituted with linear or branched alkyl groups or alkylene groups, the alkyl groups or alkylene groups overall exhibiting preferably 1 to 18 carbon atoms.

Cyclic ether alcohols such as hydroxyfurfuryl or hydroxytetrahydrofuran, nitrogen heterocyclics or sulphur heterocyclics can also be used as starting groups. Such polyalkylene glycols are disclosed in WO 01/57164, the teaching of which is herewith incorporated by reference.

Preferably, the polyalkylene glycols according to the invention have an average molecular weight (number average) of from 200 to 6000 g/mole, more preferably from 400 to 4000 g/mole, even more preferably from 1000 to 3000 g/mole and especially from 2000 to 3000 g/mole.

The polyalkylene glycols used according to the invention can be produced by reacting alcohols, including polyalcohols, as starting compounds with oxiranes such as ethylene oxide, propylene oxide and/or butylene oxide. Following the reaction, these possess only one free hydroxy group as end group. Polyalkylene glycols with only one hydroxy group are preferred over those with two free hydroxy groups. Polyalkylene glycols which, e.g. after a further etherification step, comprise no free hydroxy groups any longer are particularly preferred regarding the stability, hygroscopicity and compatibility. The alkylation of terminal hydroxyl groups leads to an increase in the thermal stability. Thus, in an especially preferred embodiment according to the present invention, the PAG base oil comprises end-capped PAG, i.e. where no free hydroxyl groups are present.

Preferably, the lubricating composition comprises at least 30 wt. % PAG base oil, preferably at least 50 wt. %, more preferably at least 70 wt. %, based on the total weight of the lubricating composition. It is even more preferred that as the base oil only (one or more) PAG base oil(s) is used.

According to a preferred embodiment of the present invention, the PAG base oil has a kinematic viscosity at 40° C. (according to ASTM D445) of from 32 to 690, preferably from 100 to 300, more preferably from 150 to 250 mm²/s.

When the base oil is a polyalkylene glycol base oil it may be used together with any of the conventionally used lubricating oils of mineral or synthetic origin. However, in one embodiment of the invention the base oil consists only of one or more polyalkylene glycol base oils.

In addition to the base oil, the lubricating grease compositions of the present invention further comprise a thickener system comprising a lithium soap of a C₁₂ to C₂₄ hydroxy carboxylic acid and an alkaline earth metal salt of a C₂ to C₁₂ dicarboxylic acid.

The amount of thickener system present in the grease is preferably from 2% to 30%, preferably from 5% to 20%, by weight of the composition.

Preferably, the lithium soap of the hydroxy C₁₂ to C₂₄ carboxylic acid is a C₁₆ to C₂₀ hydroxy carboxylic acid acid. A particularly preferred hydroxy carboxylic acid is hydroxystearic acid, for example, 9-hydroxy, 10-hydroxy, or 12-hydroxystearic acid, more preferably the latter. Ricinoleic acid which is an unsaturated form of 12-hydroxystearic acid having a double bond in the 9-10 position, can also be used. Other suitable hydroxy fatty acids include 12-hydroxybehenic acid and 10-hydroxypalmitic acid.

The C₂ to C₁₂ dicarboxylic acid is preferably a C₄ to C₁₂, more preferably C₆ to C₁₀, aliphatic dicarboxylic acid. Examples of suitable acids include oxalic, malonic, succinic, glutaric, adipic, suberic, pimelic, azelaic, dodecanedioic and sebacic acids. Azelaic and sebacic acids are especially preferred.

The alkaline earth metal present in the alkaline earth metal salt of a C₂ to C₁₂ dicarboxylic acid is preferably selected from calcium and magnesium, and mixtures thereof.

In the case where the base oil is mineral oil, particularly good lubricating performance has been observed when the alkaline earth metal is magnesium. In the case where the base oil is polyalkylene glycol, particularly good lubricating performance has been observed when the alkaline earth metal is calcium.

The C₁₂ to C₂₄ hydroxy fatty acid and the C₂ to C₁₂ aliphatic dicarboxylic acid are preferably present in a weight ratio of from 20:1 to 1:1, preferably from 10:1 to 1:1, more preferably from 8:1 to 3:1.

The grease compositions of the present invention which comprise the thickener system described hereinabove are particularly advantageous from the viewpoint of allowing a lower level of lithium metal to be used than in conventional lithium complex-based greases. The amount of lithium present in the grease compositions herein is preferably reduced by about 40 to 50 wt % compared with conventional lithium complex-based grease compositions based on lithium soaps and lithium complex soaps only and not containing any alkaline earth metal salts of C₂ to C₁₂ dicarboxylic acids.

The grease compositions herein are prepared by a process which comprises a step of making a pre-slurry comprising base oil, alkaline earth metal hydroxide or oxide and C₂ to C₁₂ dicarboxylic acid, so as to form an alkaline earth metal salt of a C₂ to C₁₂ dicarboxylic acid. In another step of the process, this pre-slurry is added to a mixture comprising base oil, lithium hydroxide and C₁₂ to C₂₄ hydroxy carboxylic acid.

Various conventional grease additives may be incorporated into the lubricating greases of the present invention, in amounts normally used in this field of application, to impart certain desirable characteristics to the grease, such as oxidation stability, tackiness, extreme pressure properties and corrosion inhibition. Suitable additives include one or more extreme pressure/antiwear agents, for example zinc salts such as zinc dialkyl or diaryl dithiophosphates, borates, substituted thiadiazoles, polymeric nitrogen/phosphorus compounds made, for example, by reacting a dialkoxy amine with a substituted organic phosphate, amine phosphates, sulphurised sperm oils of natural or synthetic origin, sulphurised lard, sulphurised esters, sulphurised fatty acid esters, and similar sulphurised materials, organo-phosphates for example according to the formula (OR)₃P═O where R is an alkyl, aryl or aralkyl group, and triphenyl phosphorothionate; one or more overbased metal-containing detergents, such as calcium or magnesium alkyl salicylates or alkylarylsulphonates; one or more ashless dispersant additives, such as reaction products of polyisobutenyl succinic anhydride and an amine or ester; one or more antioxidants, such as hindered phenols or amines, for example phenyl alpha naphthylamine; one or more antirust additives; one or more friction-modifying additives; one or more viscosity-index improving agents; one or more pour point depressing additives; and one or more tackiness agents. Solid materials such as graphite, finely divided molybdenum disulphide, talc, metal powders, and various polymers such as polyethylene wax may also be added to impart special properties.

To reduce friction levels, those skilled in the art have largely looked to using organic molybdenum-based formulations, and there are numerous proposals in patent literature of such lubricating compositions.

The present invention will now be described by reference to the following Examples:

### Examples 1 to 4 and Comparative Examples A and B

The lubricating greases of the examples below were prepared by the following procedure. A pre-manufactured slurry was prepared by mixing 10% base oil, sebacic acid, calcium hydroxide or magnesium oxide and 30 ml of water, and stirring this mixture for 20 minutes. The pre-manufactured slurry was charged to an autoclave together with 50% of the base oil, 12-hydroxystearic acid, lithium hydroxide monohydrate and 100 ml of water. The autoclave was closed and heated up to 145° C. After reaching the venting temperature the venting valve was opened and steam was released for 30 minutes. When the steam pressure was 0 bar, with the venting valve still open, heating was started up to a temperature of 215° C. After reaching a temperature of 215° C., the autoclave was cooled down with jacket cooling of 1° C./min to reach 165° C. After reaching 165° C. the remaining 50% of base oil was charged in the vessel. Then the product was cooled to 80° C. and any additives were charged in the vessel. Then the product was homogenized with a triple roll mill.

The compositions of the prepared greases are set out in Tables 1 and 2 below. Examples 1 and 2 and Comparative Example A are based on mineral oil. The grease composition according to Example 1 comprises a mixture of mineral oil base oil, lithium 12-hydroxystearate and calcium sebacate. Example 2 is the same as Example 1 except that it contains magnesium sebacate instead of calcium sebacate. Comparative Example A is the same as Examples 1 and 2 except that it comprises a mixture of lithium 12-hydroxystearate and lithium sebacate (i.e. no calcium or magnesium sebacate). Examples 3 and 4 and Comparative Example B are based on polyalkyleneglycol base oils. The grease composition according to Example 3 comprises a mixture of polyalkyleneglycol base oil, lithium 12-hydroxystearate and calcium sebacate. Example 4 is the same as Example 3 except that it contains magnesium sebacate instead of calcium sebacate. Comparative Example B is the same as Examples 3 and 4 except that it comprises a mixture of lithium 12-hydroxystearate and lithium sebacate (i.e. no calcium or magnesium sebacate).

TABLE 1
Examples 1 and 2 and Comparative Example A

| | Comparative | | |
|---|---|---|---|
| | Example A | Example 1 | Example 2 |
| Synalox 100- | 0 | 0 | 0 |
| 150B¹ | | | |
| HVI 170² | 83.82 | 83.82 | 84.13 |
| LiOH—H₂0 | 2.3 | 1.52 | 1.52 |
| Ca(OH)₂ | 0 | 0.78 | 0 |
| MgO | 0 | 0 | 0.47 |
| Sebacic acid | 1.88 | 1.88 | 1.88 |
| 12- | 10 | 10 | 10 |
| hydroxystearic | | | |
| acid | | | |
| Naugalube AMS³ | 0.5 | 0.5 | 0.5 |
| Ralox LC⁴ | 0.5 | 0.5 | 0.5 |
| Irganox L57⁵ | 0.5 | 0.5 | 0.5 |
| Valirex Zn 8.0⁶ | 0.5 | 0.5 | 0.5 |

¹polypropylene glycol homopolymer commercially available from Dow Chemicals
²mineral oil having a viscosity at 40° C. of 110 mm²s⁻¹ and a viscosity index of 95 commercially available from Shell Oil Company
³commercially available from Chimtura, USA
⁴commercially available from Raschig, Ludwigshafen, Germany
⁵commercially available from CIBA Geigy Specialties, Switzerland
⁶commercially available from Corm Van Loocke, Belgium

TABLE 2
Examples 3 and 4 and Comparative Example B

| | Comparative | | |
|---|---|---|---|
| | Example B | Example 3 | Example 4 |
| Synalox 100- | 83.82 | 83.82 | 84.10 |
| 150B¹ | | | |
| HVI 170² | 0 | 0 | 0 |
| LiOH—H₂0 | 2.3 | 1.52 | 1.52 |
| Ca(OH)₂ | 0 | 0.78 | 0 |
| MgO | 0 | 0 | 0.5 |
| Sebacic acid | 1.88 | 1.88 | 1.88 |
| 12- | 10 | 10 | 1.88 |
| hydroxystearic | | | |
| acid | | | |
| Naugalube | 0.5 | 0.5 | 0.5 |
| AMS³ | | | |
| Ralox LC⁴ | 0.5 | 0.5 | 0.5 |
| Irganox L57⁵ | 0.5 | 0.5 | 0.5 |
| Valirex Zn | 0.5 | 0.5 | 0.5 |
| 8.0⁶ | | | |

¹polypropylene glycol homopolymer commercially available from Dow Chemicals
²mineral oil having a viscosity at 40° C. of 110 mm²s⁻¹and a viscosity index of 95 commercially available from Shell Oil Company
³commercially available from Chimtura, USA
⁴commercially available from Raschig, Ludwigshafen, Germany
⁵commercially available from CIBA Geigy Specialties, Switzerland
⁶commercially available from Corm Van Loocke, Belgium

### Measurement of Lubrication Properties

The greases of Examples 1 to 4 and Comparative Examples A and B were subjected to various standard test methods in order to measure a variety of different lubrication properties. The various test methods used are listed below.
Unworked penetration was measured using DIN ISO 2137. Worked penetration was measured using DIN ISO 2137.
Worked penetration after rolltest was measured using ISO 2137/ASTM-D1831.
Worked penetration differential was measured using ISO 2137/ASTM-D1831.
Oil Bleeding was measured using DIN 51817/IP121/ASTMD6184/98

### Results

The results are shown in Tables 3 and 4 below

TABLE 3
Results from Examples 1 and 2 and Comparative
Example A

| | A | 1 | 2 |
|---|---|---|---|
| Dropping point (° C.) | 224 | 226 | 221 |
| Unworked penetration | 260 | 254 | 231 |
| (0.1 mm)⁷ | | | |
| Worked penetration | 267 | 276 | 256 |
| (0.1 mm)⁷ | | | |
| Worked penetration | 319 | 305 | 278 |
| after rolltest | | | |
| (0.1 mm)⁸ | | | |
| Worked penetration | 52 | 29 | 22 |
| differential after | | | |
| rolltest (0.1 mm)⁸ | | | |
| Worked penetration | 475 | 334 | 375 |
| after rolltest | | | |
| (24 h/80° C./10% | | | |
| water) (0.1 mm)⁹ | | | |
| Worked penetration | 208 | 58 | 120 |
| differential after | | | |
| rolltest (24 h/80 C./10% | | | |
| water) (0.1 mm)⁹ | | | |
| Oil bleeding (wt %)¹⁰ | 8.8 | 6.9 | 3.2 |
| Oil bleeding (wt %)¹¹ | 16.5 | 18.9 | 7.9 |

⁷measured at 25° C.
⁸carried out at 80° C. for 50 hours
⁹carried out at 80° C. for 24 hours in 10 wt % water
¹⁰carried out at 120° C. for 18 hours
¹¹carried out at 120° C. for 7 days

TABLE 4
Results for Examples 3 and 4 and Comparative
Example B

| | B | 3 | 4 |
|---|---|---|---|
| Dropping point (° C.) | 264 | 231 | 290 |
| Unworked penetration | 232 | 204 | 240 |
| (0.1 mm)⁷ | | | |
| Worked penetration | 240 | 216 | 274 |
| (0. 1 mm)⁷ | | | |
| Worked penetration | 281 | 279 | 295 |
| after rolltest | | | |
| (0.1 mm)⁸ | | | |
| Worked penetration | 41 | 63 | 21 |
| differential after | | | |
| rolltest (0.1 mm)⁸ | | | |
| Worked penetration | 380 | 249 | 295 |
| after rolltest | | | |
| (24 h/80° C./10% | | | |
| water) (0.1 mm)⁹ | | | |
| Worked penetration | 140 | 33 | 21 |
| differential after | | | |
| rolltest (24 h/80 C./10% | | | |
| water) (0.1 mm)⁹ | | | |
| Oil bleeding (wt %)¹⁰ | 1.8 | 0.7 | 2.6 |
| Oil bleeding (wt %)¹¹ | 4.2 | 1.6 | 7.2 |

⁷measured at 25° C.
⁸carried out at 80° C. for 50 hours
⁹carried out at 80° C. for 24 hours in 10 wt % water
¹⁰carried out at 120° C. for 18 hours
¹¹carried out at 120° C. for 7 days

### Discussion

As can be seen from Table 3 (greases based on mineral oil), the grease of Example 1 (containing lithium 12-hydroxystearate and calcium sebacate) exhibits comparable oil bleeding properties to the grease of Comparative Example A (containing lithium 12-hydroxystearate and lithium sebacate). As also can be seen from Table 3, the grease of Example 2 (containing lithium 12-hydroxystearate and magnesium sebacate) exhibits significantly reduced oil bleeding properties compared with the grease of Comparative Example A.

As can be seen from Table 4 (polyalkylene glycol based greases), the grease of Example 3 (containing lithium 12-hydroxystearate and calcium sebacate) exhibits significantly reduced oil bleeding properties compared with the grease of Comparative Example B (containing lithium 12-hydroxystearate and lithium sebacate). As also can be seen from Table 4, the grease of Example 4 (containing lithium 12-hydroxystearate and magnesium sebacate) exhibits comparable oil bleeding properties compared to Comparative Example B.

As can be seen from Table 3 the greases of Examples 1 and 2 have improved shear stability properties compared to the grease of Comparative Example A (as demonstrated by Examples 1 and 2 having a lower value than Comparative Example A for Worked penetration after rolltest and Worked Penetration differential after rolltest). As can be seen from Table 4 the greases of Examples 3 and 4 have improved shear stability properties compared to the grease of Comparative Example B (as demonstrated by Examples 3 and 4 having a lower value than Comparative Example B for Worked penetration after rolltest and Worked Penetration differential after rolltest).

As can be seen from Table 3 the greases of Examples 1 and 2 have improved water resistance properties than the grease of Comparative Example A (as demonstrated by Examples 1 and 2 having a lower value than Comparative Example A for Worked penetration after rolltest (24 h/80° C./10% water) and Worked Penetration differential after rolltest (24 h/80° C./10% water)). As can be seen from Table 4 the greases of Examples 3 and 4 have improved water resistance properties than the grease of Comparative Example B (as demonstrated by Examples 3 and 4 having a lower value than Comparative Example B for Worked Penetration after rolltest (24 h/80° C./10% water) and Worked Penetration differential after rolltest (24 h/80° C./10% water)).

1. Lubricating grease composition comprising:
a base oil; and
a thickener system comprising (a) a lithium soap of a C₁₂ to C₂₄ hydroxycarboxylic acid and (b) an alkaline earth metal salt of a C₂ to C₁₂ dicarboxylic acid.

2. A Lubricating composition according to claim 1 wherein the C₁₂ to C₂₄ hydroxycarboxylic acid is a C₁₆ to C₂₀ hydroxycarboxylic acid.

3. A Lubricating composition according to claim 1 wherein the C₁₂ to C₂₄ hydroxycarboxylic acid is 12-hydroxystearic acid.

4. A Lubricating composition according to claim 1 wherein the C₂ to C₁₂ dicarboxylic acid is selected from azelaic acid, sebacic acid, oxalic acid, malonic acid, succinic acid, glutaric acid, adipic acid, suberic acid, pimelic acid and dodecanedioic acid and mixtures thereof.

5. A Lubricating composition according to claim 1 wherein the C₂ to C₁₂ dicarboxylic acid is selected from azelaic acid, sebacic acid and mixtures thereof.

6. A Lubricating composition according to claim 1 wherein the alkaline earth metal is selected from calcium, magnesium and mixtures thereof.

7. A Lubricating composition according to claim 1 wherein the base oil is selected from a mineral oil base oil, a polyalkyleneglycol base oil, and mixtures thereof.

8. A process for preparing the lubricating composition according to claim 1 comprising the steps of:
(i) making a pre-slurry comprising base oil, alkaline earth metal hydroxide or oxide and C₂ to C₁₂ dicarboxylic acid, so as to form an alkaline earth metal salt of a C₂ to C₁₂ dicarboxylic acid; and
(ii) adding the pre-slurry prepared in step (i) to a mixture comprising base oil, lithium hydroxide and C₁₂ to C₂₄ hydroxy carboxylic acid.

9. A process comprising using a lubricating grease composition as described in claim 1 in a dual mass flywheel application.

10. A process comprising measuring the oil bleeding characteristic of a lubricating composition as described in claim 1.

11. A process comprising measuring the water resistance characteristic of a lubricating composition as claimed in claim 1.

12. A process comprising measuring the shear stability of a lubricating composition as claimed in claim 1.

13. A Lubricating composition according to claim 2 wherein the C₁₂ to C₂₄ hydroxycarboxylic acid is 12-hydroxystearic acid.

14. A Lubricating composition according to claim 13 wherein the C₂ to C₁₂ dicarboxylic acid is selected from azelaic acid, sebacic acid, oxalic acid, malonic acid, succinic acid, glutaric acid, adipic acid, suberic acid, pimelic acid and dodecanedioic acid and mixtures thereof.

15. A Lubricating composition according to claim 14 wherein the C₂ to C₁₂ dicarboxylic acid is selected from azelaic acid, sebacic acid and mixtures thereof.

16. A Lubricating composition according to claim 15 wherein the alkaline earth metal is selected from calcium, magnesium and mixtures thereof.

17. A Lubricating composition according to claim 16 wherein the base oil is selected from a mineral oil base oil, a polyalkyleneglycol base oil, and mixtures thereof.
